(19) United States
(12) Patent Application Publication
(10) Pub. No.: US 2014/0186325 A1
(43) Pub. Date: Jul. 3, 2014

(54) Methods of Cancer Treatment and Prevention Through the Modulation of SIRT4 Activity

(71) Applicants: Marcia C. Haigis, Winchester, MA (US); Seungmin Jeong, Brookline, MA (US)

(72) Inventors: Marcia C. Haigis, Winchester, MA (US); Seungmin Jeong, Brookline, MA (US)

(73) Assignee: President and Fellows of Harvard College, Cambridge, MA (US)

(21) Appl. No.: 14/125,175

(22) PCT Filed: Jun. 7, 2012
(86) PCT No.: PCT/US12/41304

§ 371 Date: Mar. 13, 2014

Related U.S. Application Data
(60) Provisional application No. 61/495,758, filed on Jun. 10, 2011.

(51) Int. Cl. A61K 38/45; G01N 33/574; C12Q 1/68

(52) U.S. Cl. CPC A61K 38/45; C12Q 1/6886; G01N 33/57484
USPC 424/94.5; 514/44 R; 435/375; 435/6.11; 436/501

(57) ABSTRACT

The present invention relates to methods of preventing or treating cancer through the use of agents that enhance the activity or expression of SIRT4.

### RELATED APPLICATIONS

This application claims the benefit of priority to U.S. Provisional Patent Application Ser. No. 61/495,758, filed March Jun. 10, 2011, which is hereby incorporated by reference in its entirety.

### BACKGROUND

Sirtuins are a highly conserved family of NAD⁺-dependent deacetylases and ADP-ribosylases with various roles in metabolism, stress resistance and longevity. In mammalian cells there are seven sirtuins (SIRT1-7) that are localized in the nucleus (SIRT1, 6 and 7), mitochondria (SIRT3, 4, and 5) and cytoplasm (SIRT2). SIRT4 has been shown to repress glutamate dehydrogenase through ADP-ribosylation and/or deacetylation. SIRT4 also represses insulin secretion in the pancreas and fatty acid oxidation in the liver.

In many cancer cells, glutamine is the primary mitochondrial substrate and is required for maintenance of mitochondrial function and integrity. Glutamine is an essential metabolite for proliferating cells. Cancer cells show high levels of glutamine metabolism and glutamine is required for oncogenic transformation. Tumor cells with enhanced expression of the c-Myc oncogene can not survive in the absence of glutamine.

Methods of modulating the level of glutamine metabolism in a cell therefore offer great promise for the prevention and/or treatment of cancer.

### SUMMARY

In some embodiments the present invention relates to a method of treating or preventing cancer in a subject (e.g., a mammalian subject, such as a human subject) that includes administering to the subject an agent that increases SIRT4 activity. In some embodiments the agent is a small molecule, a polypeptide or a nucleic acid. In certain embodiments the agent increases the expression of SIRT4 protein or SIRT4 mRNA. In some embodiments the agent inhibits glutamine consumption. In some embodiments the subject had or is predisposed to cancer (e.g., lung cancer).

In certain embodiments, the present invention relates to a method of inhibiting the proliferation of a tumor cell (e.g., a lung tumor cell) that includes contacting the tumor cell with an agent the increases SIRT4 protein activity. In some embodiments the tumor is in a subject (e.g., a mammalian subject, such as a human subject). In some embodiments the tumor cell has reduced SIRT4 activity and/or comprises one or two mutated SIRT4 genes. In some embodiments the agent is a small molecule, a polypeptide or a nucleic acid. In certain embodiments the agent increases the expression of SIRT4 protein or SIRT4 mRNA. In some embodiments the agent inhibits glutamine consumption.

In some embodiments the present invention relates to a method of determining whether a subject is predisposed to cancer that includes the steps of obtaining a tissue sample from the subject and determining whether the tissue sample comprises a mutated SIRT4 gene, wherein the presence of a mutated SIRT4 gene indicates that the subject is predisposed to cancer. In some embodiments the tissue sample is blood sample, a tissue biopsy sample, a cheek swab sample, a hair sample, a saliva sample or a skin sample. In certain embodiments the method also includes determining whether the tissue sample comprises a second mutated SIRT4 gene and/or determining whether the tissue sample comprises an unmutated SIRT4 gene. In some embodiments the presence of a mutated or unmutated SIRT4 gene is determined by sequencing at least part of the SIRT4 gene or by contacting the SIRT4 gene (or an amplicon thereof) with a nucleic acid probe.

### BRIEF DESCRIPTION OF THE DRAWINGS

FIG. 1 shows that glutamine metabolism is repressed in response to DNA damage in both HepG2 cells and MEF cells.

FIG. 2 shows that glutamine metabolism is repressed in response to DNA damage in transformed MEF cells.

FIG. 3 shows that glutamine entry into the TCA cycle is repressed in response to DNA damage.

FIG. 4 shows that SIRT4 mRNA expression (2A) and protein expression (2B) are induced by genotoxic stress in MEF cells and HEK293T cells.

FIG. 5 shows that SIRT4 overexpression (OE) inhibits glutamine metabolism in HEK293T cells and in HeLa cells.

FIG. 6 shows that SIRT4 regulates glutamine metabolism in HepG2 cells, HeLa cells and PC3 cells.

FIG. 7 shows that SIRT4 regulates glutamine metabolism in HEK293T cells.

FIG. 8 shows that SIRT4 regulates glutamine metabolism in response to genotoxic stress in HepG2 cells.

FIG. 9 shows that SIRT4 regulates glutamine metabolism in response to genotoxic stress in immortalized MEF cells.

FIG. 10 shows that glutamine consumption is increased in SIRT4 KO MEF cells.

FIG. 11 shows that SIRT4 and p21 expression is induced by genotoxic stress in HEK293T cells and HepG2 cells.

FIG. 12 shows that SIRT4 inhibits mitochondrial metabolism in HepG2 cells.

FIG. 13 shows that SIRT4 regulates mitochondrial glutamine metabolism.

FIG. 14 shows that SIRT4 protects against DNA damage-induced apoptotic cell death in HepG2 cells.

FIG. 15 shows that SIRT4 protects against DNA damage-induced apoptotic cell death.

FIG. 16 shows that SIRT4 protects against DNA damage-induced apoptotic cell death in MEF cells.

FIG. 17 shows that SIRT4 inhibits tumor cell growth by repressing mitochondrial glutamine metabolism.

FIG. 18 shows that E1A and Ras expressing SIRT4 knock-out MEF cells become transformed at a higher frequency than E1A and Ras expressing SIRT4 wild-type MEF cells.

FIG. 19 shows that E1A and Ras expressing SIRT4 knock-out MEF cells acquire a tumorigenic phenotype at a higher frequency than E1A and Ras expressing SIRT4 wild-type MEF cells.

FIG. 20 shows that glutamine metabolism is essential for the transformation of SIRT4 knockout MEF cells.

FIG. 21 shows that SIRT4 has a tumor suppressive function.

FIG. 22 shows that tumorogenesis occurs more frequently in SIRT4 knockout mice than in SIRT4 wild-type mice.

FIG. 23 shows that SIRT4 expression is decreased in several human cancers.

### DETAILED DESCRIPTION

Cancer often results from uncontrolled cell division in cells with damaged DNA. As described herein, the instant invention relates to the discovery that genotoxic stress, such as is caused by DNA damage, results in the elevated expression of SIRT4. Without being bound by theory, this elevated SIRT4 expression inhibits glutamine consumption and mitochondrial metabolism, resulting in metabolic pause and cell cycle arrest. In the absence of the elevated SIRT4 an elevated frequency of cells will proliferate despite the DNA damage, resulting in an increased cellular transformation and tumorogenesis. Thus, as described herein, agents that increase SIRT4 activity and/or expression are useful for the prevention and/or treatment of cancer and individuals who have reduced SIRT4 expression and/or activity (e.g., those for whom one or both of their SIRT4 genes are mutated) are predisposed to cancer.

The embodiments and practices of the present invention, other embodiments, and their features and characteristics, will be apparent from the description, figures and claims that follow, with all of the claims hereby being incorporated by this reference into this Description.

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The terms “agent” are used herein to denote a chemical compound, a small molecule, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “binding” or “interacting” refers to an association, which may be a stable association, between two molecules, e.g., between a polypeptide and a binding partner or agent, e.g., small molecule, due to, for example, electrostatic, hydrophobic, ionic and/or hydrogen-bond interactions under physiological conditions.

“Biologically active portion of SIRT4” refers to a portion of SIRT4 protein having a biological activity, such as the ability to deacetylate or ADP-ribosylate. Biologically active portions of a SIRT4 may comprise the core domain of SIRT4.

As used herein, the term “cancer” includes, but is not limited to, solid tumors and blood borne tumors. The term cancer includes diseases of the skin, tissues, organs, bone, cartilage, blood and vessels. The term “cancer” further encompasses primary and metastatic cancers.

The term “control” includes any portion of an experimental system designed to demonstrate that the factor being tested is responsible for the observed effect, and is therefore useful to isolate and quantify the effect of one variable on a system. A control includes a “reference sample” as described herein.

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination there of, which (1) is not associated with the cell in which the “isolated nucleic acid” is found in nature, or (2) is operably linked to a polynucleotide to which it is not linked in nature.

The term “mammal” is known in the art, and exemplary mammals include humans, primates, bovines, porcines, canines, felines, and rodents (e.g., mice and rats).

The term “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity or process. In certain instances, such regulation may be contingent on the occurrence of a specific event, such as activation of a signal transduction pathway, and/or may be manifest only in particular cell types.

A “modulator” may be a polypeptide, nucleic acid, macromolecule, complex, molecule, small molecule, compound, species or the like (naturally-occurring or non-naturally-occurring), or an extract made from biological materials such as bacteria, plants, fungi, or animal cells or tissues, that may be capable of causing modulation. Modulators may be evaluated for potential activity as inhibitors or activators (directly or indirectly) of a functional property, biological activity or process, or combination of them, (e.g., agonist, partial antagonist, partial agonist, inverse agonist, antagonist, anti-microbial agents, inhibitors of microbial infection or proliferation, and the like) by inclusion in assays. In such assays, many modulators may be screened at one time. The activity of a modulator may be known, unknown or partially known.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

A “patient” or “subject” refers to either a human or a non-human animal.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” is art-recognized and refers to the relatively non-toxic, inorganic and organic acid addition salts of compounds, including, for example, those contained in compositions described herein.

The term “SIRT4-activating compound” or “agent that increases SIRT4 activity” refers to an agent that increases the level of SIRT4 protein and/or increases at least one activity of a SIRT4 protein. In an exemplary embodiment, a SIRT4-activating compound may increase at least one biological activity of a SIRT4 protein by at least about 10%, 25%, 50%, 75%, 100%, or more. Exemplary biological activities of SIRT4 proteins include inhibition of glutamine consumption and inhibition of glutamine metabolism.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

The phrases “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### SIRT4 Proteins

As used herein, the term “SIRT4” or “SIRT4 protein” refers to proteins, e.g., eukaryotic proteins, e.g., mammalian proteins, comprising a mitochondrial protein having ADP-ribosyl transfer case activity, as well as functional domains, fragments (e.g., functional fragments), e.g., fragments of at least 8 amino acids, e.g., at least 8, 18, 28, 64, 128, 150, 180, 200, 220, 240, 260, or 280 amino acids, and variants thereof. Exemplary functional fragments of SIRT4 can, for example, have ADP-ribosyltransferase activity and/or the ability to interact with a SIRT4 binding partner. Exemplary SIRT4 proteins include those designated GenBank NM_{—}012240 (human SIRT4) and XM_{—}485674 (mouse SIRT4). Homologs of SIRT4 proteins will share 60%, 80%, 85%, 90%, 95%, 98%, 99% sequence identity to a known SIRT4 protein and feature an SIRT4 activity, e.g., ADP ribosylation, inhibition of fatty acid oxidation, downregulation of glutamate dehydrogenase, inhibition of glutamine consumption, inhibition of mitochondrial metabolism. Eukaryotic SIRT4 proteins may be localized, e.g., to mitochondria. Variants of SIRT4 proteins can be produced by standard means, including site-directed and random mutagenesis.

In certain embodiments, a protein described herein is further linked to a heterologous polypeptide, e.g., a polypeptide comprising a domain which increases its solubility and/or facilitates its purification, identification, detection, and/or structural characterization. A protein described herein may be linked to at least 2, 3, 4, 5, or more heterologous polypeptides. Polypeptides may be linked to multiple copies of the same heterologous polypeptide or may be linked to two or more heterologous polypeptides. The fusions may occur at the N-terminus of the polypeptide, at the C-terminus of the polypeptide, or at both the N- and C-terminus of the polypeptide. It is also within the scope of the invention to include linker sequences between a protein described herein and the fusion domain in order to facilitate construction of the fusion protein or to optimize protein expression or structural constraints of the fusion protein.

In another embodiment, a protein may be modified so that its rate of traversing the cellular membrane is increased. For example, the polypeptide may be fused to a second peptide which promotes “transcytosis,” e.g., uptake of the peptide by cells. The peptide may be a portion of the HIV transactivator (TAT) protein, such as the fragment corresponding to residues 37-62 or 48-60 of TAT, portions which have been observed to be rapidly taken up by a cell in vitro (Green and Loewenstein, (1989) Cell 55:1179-1188). Alternatively, the internalizing peptide may be derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is coupled. Thus, the polypeptide may be fused to a peptide consisting of about amino acids 42-58 of Drosophila antennapedia or shorter fragments for transcytosis (Derossi et al. (1996) J Biol Chem 271:18188-18193; Derossi et al. (1994) J Biol Chem 269:10444-10450; and Perez et al. (1992) J Cell Sci 102:717-722). The transcytosis polypeptide may also be a non-naturally-occurring membrane-translocating sequence (MTS), such as the peptide sequences disclosed in U.S. Pat. No. 6,248,558.

### SIRT4 Nucleic Acids

Nucleic acids encoding any of the polypeptides described herein are also provided herein. A nucleic acid may further be linked to a promoter and/or other regulatory sequences, as further described herein. Exemplary nucleic acids are those that are at least about 80%, 85%, 90%, 95%, 98%, 99% or 100% identical to a nucleotide sequence provided herein or a fragment thereof, such as nucleic acid sequence encoding the protein fragments described herein. Nucleic acids may also hybridize specifically, e.g., under stringent hybridization conditions, to a nucleic acid described herein or a fragment thereof.

Nucleic acids, e.g., those encoding a protein of interest or functional homolog thereof, can be delivered to cells in culture, ex vivo, and in vivo. The cells can be of any type including without limitation cancer cells, stem cells, neuronal cells, myocytes, and non-neuronal cells. The delivery of nucleic acids can be by any technique known in the art including viral mediated gene transfer, liposome mediated gene transfer, direct injection into a target tissue, organ, or tumor, injection into vasculature which supplies a target tissue or organ.

Polynucleotides can be administered in any suitable formulations known in the art. These can be as virus particles, as naked DNA, in liposomes, in complexes with polymeric carriers, etc. Polynucleotides can be administered to the arteries which feed a tissue or tumor. They can also be administered to adjacent tissue, whether tumor or normal, which could express the protein.

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

A polynucleotide of interest can also be combined with a condensing agent to form a gene delivery vehicle. The condensing agent may be a polycation, such as polylysine, polyarginine, polyornithine, protamine, spermine, spermidine, and putrescine. Many suitable methods for making such linkages are known in the art.

In an alternative embodiment, a polynucleotide of interest is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell which has internalized the liposome, thereby releasing its contents into the cytoplasm. Prior to interaction with the surface of a cell, however, the liposome membrane acts as a relatively impermeable barrier that sequesters and protects its contents, for example, from degradative enzymes. Additionally, because a liposome is a synthetic structure, specially designed liposomes can be produced which incorporate desirable features. See Stryker, Biochemistry, pp. 236-240, 1975 (W.H. Freeman, San Francisco, Calif.); Soak et al., Biochip. Biopsy's. Acta 600:1, 1980; Bayer et al., Biochip. Biopsy's. Acta. 550:464, 1979; Rivnay et al., Meth. Enzymol. 149:119, 1987; Wang et al., PROC. NATL. ACAD. SCI. U.S.A. 84: 7851, 1987, Plant et al., Anal. Biochem. 176:420, 1989, and U.S. Pat. No. 4,762,915. Liposomes can encapsulate a variety of nucleic acid molecules including DNA, RNA, plasmids, and expression constructs comprising growth factor polynucleotides such those disclosed in the present invention.

Liposomal preparations for use in the present invention include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et al., Proc. Natl. Acad. Sci. USA 86:6077-6081, 1989), and purified transcription factors (Debs et al., J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. See also Felgner et al., Proc. Natl. Acad. Sci. USA 91: 5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Soak et al., Proc. Natl. Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

One or more polypeptide (e.g., a SIRT4 protein, or a polypeptide that modulates SIRT4 activity) of interest may be encoded by a single nucleic acid. Alternatively, separate nucleic acids may encode different protein or nucleic acids of interest. Different species of nucleic acids may be in different forms; they may use different promoters or different vectors or different delivery vehicles. Similarly, the same protein or nucleic acid of interest may be used in a combination of different forms.

### Modulators of SIRT4

Certain embodiments of the present invention relate to methods of preventing or treating cancer. These methods involve administering an agent that increases the activity and/or expression of SIRT4. Agents which may be used to increase the activity of SIRT4 include nucleic acids, proteins, peptides and small molecules.

Any agent that increases the activity of SIRT4 can be used to practice certain methods of the invention. Such agents can be those described herein, those known in the art, or those identified through routine screening assays (e.g. the screening assays described herein).

In certain embodiments, the agent increases the activity or expression of SIRT4. Such molecules are useful, for example, in methods of treating cancer, including solid tumors. SIRT4-activating agents can include, for example, SIRT4 proteins or polypeptides, SIRT4 nucleic acids, and small molecule activators of SIRT4.

In some embodiments, assays used to identify agents useful in the methods of the present invention include a reaction between SIRT4 and one or more assay components. The other components may be either a test compound (e.g. the potential agent), or a combination of test compounds and a natural binding partner or target of SIRT4 (e.g. glutamate dehydrogenase (GDH), adenine nucleotide transporter (ANT) or insulin-degrading enzyme (IDE)). Agents identified via such assays, such as those described herein, may be useful, for example, for preventing or treating cancer. Exemplary methods for identifying activators of SIRT4 activity are provided, for example, in published U.S. patent application US2011/0098190.

Agents useful in the methods of the present invention may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

Agents useful in the methods of the present invention may be identified, for example, using assays for screening candidate or test compounds which modulate the activity of SIRT4 or a biologically active portion thereof on SIRT4 substrates (e.g., GDH, ANT or IDE). For example, candidate or test compounds can be screened for the ability to modulate the protein ADP-ribosyltransferase enzymatic activity of SIRT4.

In another embodiment, agents useful in the methods of the invention may be identified using assays for screening candidate or test compounds which bind to SIRT4 or a biologically active portion thereof. Determining the ability of the test compound to directly bind to SIRT4 can be accomplished, for example, by coupling the compound with a radioisotope or enzymatic label such that binding of the compound to SIRT4 can be determined by detecting the labeled compound in a complex. For example, compounds can be labeled with ¹²⁵I, ³⁵S, ¹⁴C or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

Agents useful in the methods of the invention may also be identified, for example, using assays known in the art that identify compounds which enhance interactions between SIRT4 and a substrate and/or binding partners (e.g. GDH, ANT or IDE).

Enhancers of SIRT4 expression may also be identified, for example, using methods wherein a cell is contacted with a candidate compound and the expression of SIRT4 mRNA or protein is determined. The level of expression of mRNA or protein in the presence of the candidate compound is compared to the level of expression of mRNA or protein in the absence of the candidate compound. The candidate compound can then be identified as an enhancer of SIRT4 expression if the expression of SIRT4 is greater in the presence of the candidate compound than in its absence.

### Pharmaceutical Compositions

Pharmaceutical compositions of the invention include any activator of SIRT4 activity and/or expression (e.g., any small molecule, protein, polypeptide or polynucleotide that activates the activity or expression of SIRT4), or a pharmaceutically acceptable salt thereof, and a pharmaceutically acceptable carrier or vehicle. Pharmaceutical compositions of the invention that include agents that increase SIRT4 activity or expression are useful for treating cancers (e.g., solid tumors).

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, the pharmaceutical compositions of the invention are useful for the prevention or treatment of cancer. Such compositions may comprise one or more agents that increase SIRT4 activity and/or expression and a second chemotherapeutic agent.

The term chemotherapeutic agent includes, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alfa, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

Further, the following drugs may also be used in combination with an antineoplastic agent, even if not considered antineoplastic agents themselves: dactinomycin; daunorubicin HCl; docetaxel; doxorubicin HCl; epoetin alfa; etoposide (VP-16); ganciclovir sodium; gentamicin sulfate; interferon alfa; leuprolide acetate; meperidine HCl; methadone HCl; ranitidine HCl; vinblastin sulfate; and zidovudine (AZT). For example, fluorouracil has recently been formulated in conjunction with epinephrine and bovine collagen to form a particularly effective combination.

Still further, the following listing of amino acids, peptides, polypeptides, proteins, polysaccharides, and other large molecules may also be used: interleukins 1 through 18, including mutants and analogues; interferons or cytokines, such as interferons α, β, and γ; hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; anti-angiogenesis factors; antigenic materials; and pro-drugs.

Chemotherapeutic agents for use with the compositions and methods of treatment described herein include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In another embodiment, the composition of the invention may comprise other biologically active substances, including therapeutic drugs or pro-drugs, for example, other chemotherapeutic agents, scavenger compounds, antibiotics, anti-virals, anti-fungals, anti-inflammatories, vasoconstrictors and anticoagulants, antigens useful for cancer vaccine applications or corresponding pro-drugs.

Exemplary scavenger compounds include, but are not limited to thiol-containing compounds such as glutathione, thiourea, and cysteine; alcohols such as mannitol, substituted phenols; quinones, substituted phenols, aryl amines and nitro compounds.

Various forms of the chemotherapeutic agents and/or other biologically active agents may be used. These include, without limitation, such forms as uncharged molecules, molecular complexes, salts, ethers, esters, amides, and the like, which are biologically active.

In certain embodiments, the pharmaceutical compositions of the invention are useful for the reduction of damage to tissues or organs exposed to a hypoxia. Tissues and organs are often exposed to hypoxic conditions during a stroke, a myocardial infarction or a peripheral vascular disease. Such compositions may comprise one or more agents that decrease SIRT4 activity and/or expression and a second therapeutic agent. Examples of therapeutic agents that can be combined with agents that decreases SIRT4 activity and/or expression include, for example, alteplase, aspirin, clopidogrel, dipyridamole, morphine, nitroglycerin, statins and tissue plasminogen activator.

### Exemplary Methods of Treatment, Prevention and Diagnosis of Disease

Provided herein are methods of treatment, prevention and/or diagnosis of conditions and diseases (e.g., cancer) that can be improved by increasing the activity of SIRT4. In some embodiments, the present invention provides therapeutic methods of treating cancer, including a cancerous tumor (e.g., a solid tumor) comprising administering to a subject, (e.g., a subject in need thereof), an effective amount of an agent that increases the expression and/or activity of SIRT4. In some embodiments, the present invention provides methods of identifying a subject predisposed to cancer comprising determining whether the subject carries one or more mutated SIRT4 genes (e.g. a SIRT4 gene that does not encode a fully functional SIRT4 protein).

The pharmaceutical compositions of the present invention may be delivered by any suitable route of administration, including orally, nasally, as by, for example, a spray, rectally, intravaginally, parenterally, intracisternally and topically, as by powders, ointments or drops, including buccally and sublingually. In certain embodiments the pharmaceutical compositions are delivered generally (e.g., via oral or parenteral administration). In certain other embodiments the pharmaceutical compositions are delivered locally through direct injection into a tumor or tumor's blood supply (e.g., arterial or venous blood supply).

In certain embodiments, the methods of treatment of the present invention comprise administering an agent that increases the activity or expression of SIRT4 in conjunction with a second therapeutic agent to the subject. Such methods may comprise administering pharmaceutical compositions comprising an agent that increases the activity or expression of SIRT4 in conjunction with one or more chemotherapeutic agents and/or scavenger compounds, including chemotherapeutic agents described herein, as well as other agents known in the art.

Conjunctive therapy includes sequential, simultaneous and separate, or co-administration of the active compound in a way that the therapeutic effects of the first agent administered have not entirely disappeared when the subsequent agent is administered. In certain embodiments, the second agent may be co-formulated with the first agent or be formulated in a separate pharmaceutical composition.

In some embodiments, the subject pharmaceutical compositions of the present invention will incorporate the substance or substances to be delivered in an amount sufficient to deliver to a patient a therapeutically effective amount of an incorporated therapeutic agent or other material as part of a prophylactic or therapeutic treatment. The desired concentration of the active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

In certain embodiments, the present invention provides therapeutic methods of treating, preventing or diagnosing cancer, including a cancerous tumor (e.g., a solid tumor) in a subject in need thereof. A subject in need thereof may include, for example, a subject who has been diagnosed with a tumor, including a pre-cancerous tumor, a cancer, or a subject who has been treated, including subjects that have been refractory to the previous treatment.

In some embodiments, the methods of the instant invention are used to determine whether a subject is predisposed to cancer. As described herein, a subject who carries one or more mutated SIRT4 genes is predisposed to cancer. In some embodiments the present invention relates to a method of determining whether a subject is predisposed to cancer that includes the steps of obtaining a tissue sample from the subject and determining whether the tissue sample comprises a mutated SIRT4 gene, wherein the presence of a mutated SIRT4 gene indicates that the subject is predisposed to cancer. In some embodiments the tissue sample is blood sample, a tissue biopsy sample, a cheek swab sample, a hair sample, a saliva sample or a skin sample. In certain embodiments the method also includes determining whether the tissue sample comprises a second mutated SIRT4 gene and/or determining whether the tissue sample comprises an unmutated SIRT4 gene. In some embodiments the presence of a mutated or unmutated SIRT4 gene is determined by sequencing at least part of the SIRT4 gene or by contacting the SIRT4 gene (or an amplicon thereof) with a nucleic acid probe.

As described herein, a subject with reduced expression or activity of SIRT4 protein indicative of a subject predisposed to cancer. The level of SIRT4 protein and/or RNA can be determined using any method known in the art. For example, in certain embodiments the level of SIRT4 protein is determined using antibodies or antigen binding fragments thereof that bind specifically to SIRT4. In some embodiments the antibodies are either directly or indirectly labeled with a detectable moiety. In some embodiments the SIRT4 RNA level is determined using a nucleic acid amplification assay, such as, for example, a PCR, SDA, TMA or NASBA based assay. In some embodiments the SIRT4 RNA level is determined using a nucleic acid probe that binds specifically to SIRT4 RNA or an amplification product produced from SIRT4 RNA.

The methods of the present invention may be used to treat, prevent or diagnose any cancerous or pre-cancerous tumor. In certain embodiments, the cancerous tumor is a lung tumor. In certain embodiments, the tumor has reduced expression of SIRT4 protein or mRNA relative to non-tumor tissue (e.g., a non-tumor tissue of the same tissue type as the tumor). Tumors may be located, for example, in a tissue selected from brain, colon, urogenital, lung, renal, prostate, pancreas, liver, esophagus, stomach, hematopoietic, breast, thymus, testis, ovarian, skin, bone marrow and/or uterine tissue.

In some embodiments, methods and compositions of the present invention may be used to treat, prevent or diagnose any cancer. Cancers that may treated, prevented or diagnosed by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

All publications, including patents, applications, and GenBank Accession numbers mentioned herein are hereby incorporated by reference in their entirety as if each individual publication or patent was specifically and individually indicated to be incorporated by reference. In case of conflict, the present application, including any definitions herein, will control.

The invention now being generally described, it will be more readily understood by reference to the following examples, which are included merely for purposes of illustration of certain aspects and embodiments of the present invention, and are not intended to limit the invention.

### EXEMPLIFICATION

### Example 1

### Glutamine Metabolism is Repressed in Response to DNA Damage

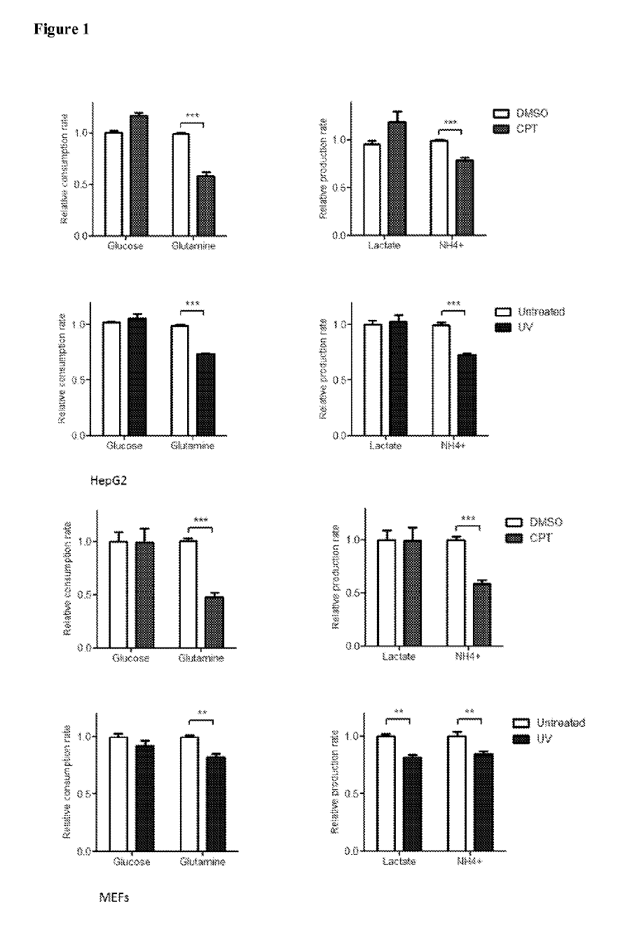

The relationship between glutamine metabolism and DNA damage was investigated. HepG2 cells and MEF cells were treated with either 14 μM camptothecin (CPT) for 12 hours or 50 J/m² ultra-violet (UV) radiation, both of which are known to induce DNA damage in cells. Glucose, glutamine, lactate and ammonia (NH₄⁺) levels in culture media were measured using the BioProfile FLEX analyzer (Nova Biomedical) according to the manufacturer's instructions. As shown in FIG. 1, treatment of both cell types with either CPT or UV resulted in significant reductions in glutamine consumption and NH₄⁺ production. These results indicate that glutamine metabolism is repressed in response to DNA damage.

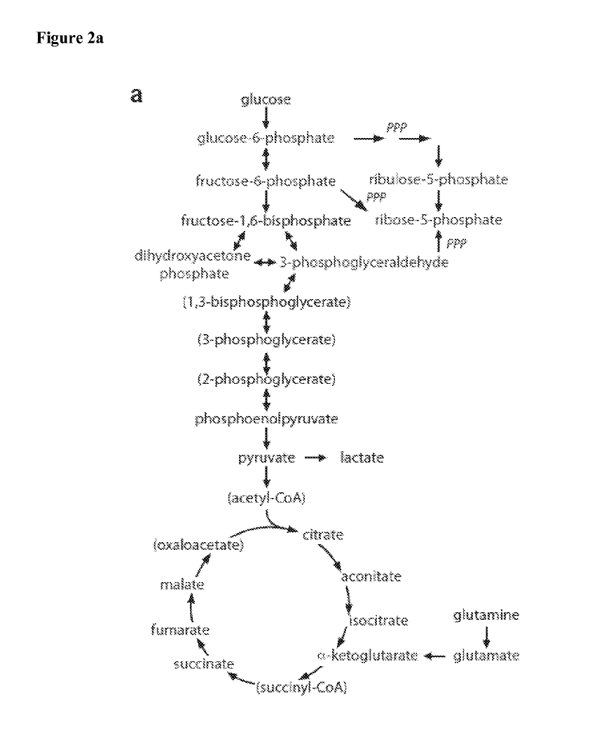

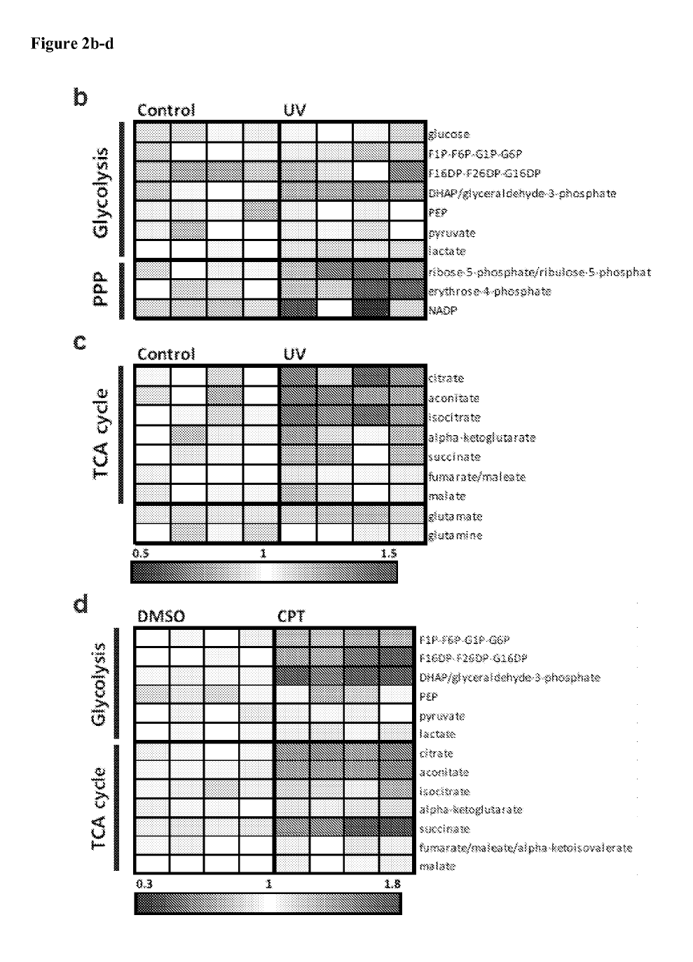

To examine further how glutamine metabolism is regulated by genotoxic stress, a global metabolomic analysis of transformed MEFs was performed before and after 20 J/m² UV treatment. A decrease in measured TCA cycle intermediates occurred after UV exposure. (FIG. 2a-c), indicating that cells reduced glutamine-derived anaplerosis following DNA damage. Moreover, a similar metabolic shift occurred in HepG2 cells treated with 14 μM CPT for 12 hr (FIG. 2d). Taken together, these data indicate that cells coordinate metabolic reprogramming upon DNA damage to limit carbon entry into the TCA cycle.

### Example 2

### Glutamine Entry into the TCA cycle is Repressed in Response to DNA Damage

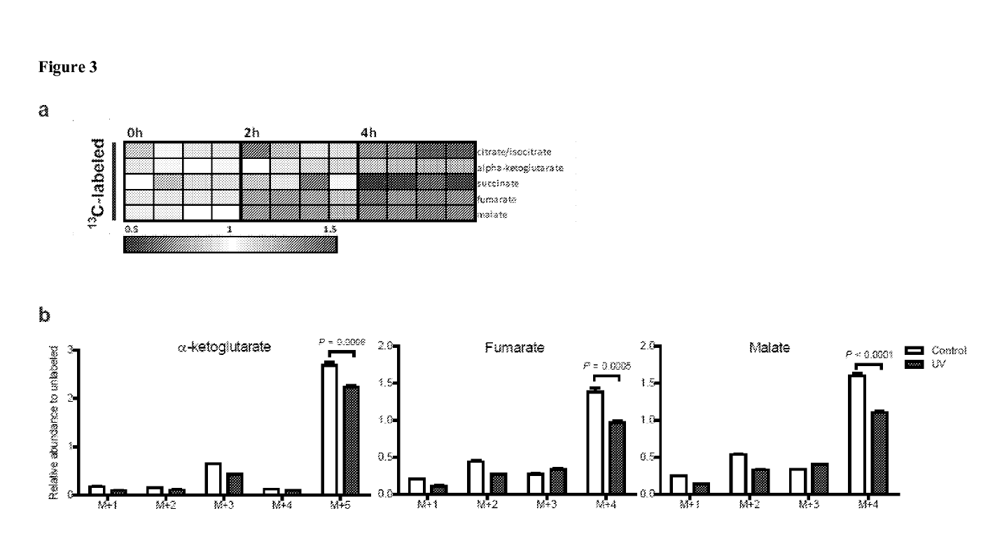

A time-course tracer study was performed to directly monitor the incorporation of [U-¹³C]glutamine into TCA cycle intermediates at 0, 2 and 4 hr after 20 J/m² UV treatment. After UV exposure, cells had reduced contribution of glutamine to TCA cycle intermediates in a time-dependent manner (FIG. 3a). Moreover, the majority of the labeled fumarate and malate contained four carbon atoms derived from ¹³C-labeled glutamine, indicating that most glutamine was used in the non-reductive direction towards succinate, fumarate and malate production (FIG. 3b). Taken together, the metabolic flux analysis demonstrate that DNA damaging stress causes cells to reduce mitochondrial glutamine anaplerosis, thus limiting the critical refueling of carbons into the TCA cycle.

### Example 3

### SIRT4 is Induced by Genotoxic Stress

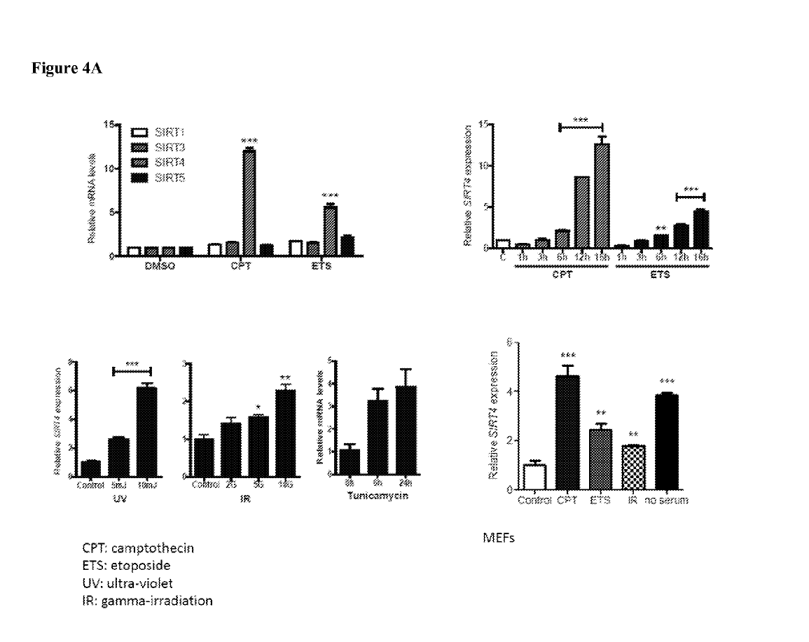

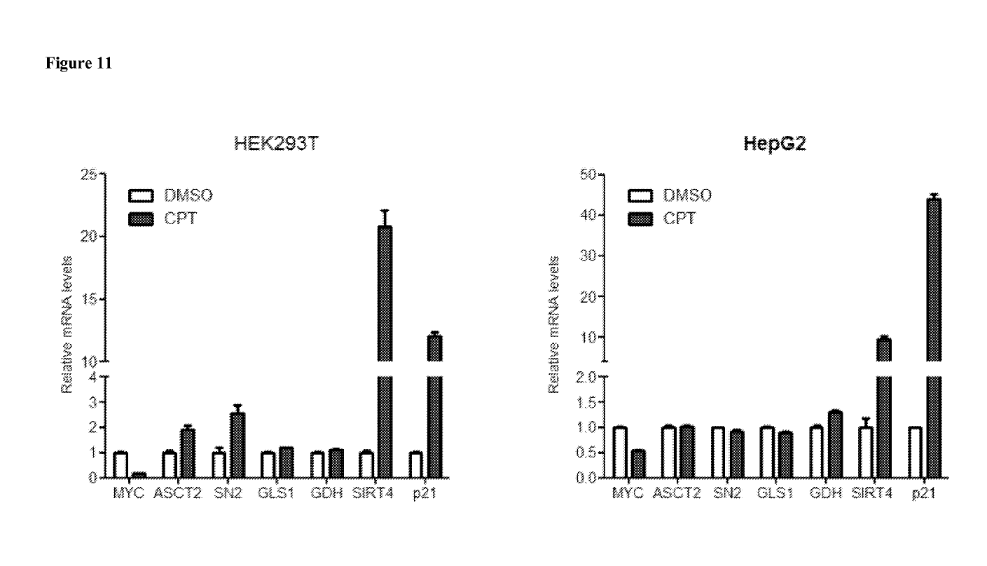

The relationship between sirtuin expression and DNA damage was next investigated. HEK293T cell and MEF cells were treated with 14 μM CPT, 25 μM etoposide (ETS), UV radiation, gamma-irradiation (IR) or 48 μM tunicamycin and the expression level of the SIRT1, SIRT3, SIRT4 and SIRT5 was determined. As shown in FIG. 4A, treatment with DNA damaging agents significantly induced expression of SIRT4, but not SIRT1, SIRT3 or SIRT5. The level of SIRT4 expression correlated with the length of time the HEK293T cells were exposed to CPT, ETS or Tunicamycin (FIG. 4A). The level of SIRT4 expression in HEK293 cells also correlated with the amount of UV radiation or IR to which cells were exposed (FIG. 4A). DNA damaging agents also induced elevated levels of SIRT4 expression in MEF cells (FIG. 4A) and Hep2G cells (FIG. 11).

To determine whether exposure to DNA damaging agents caused cells to express elevated levels of SIRT4 protein, as well as mRNA, HEK293T cells and MEF cells were treated with CPT or ETS and SIRT4 protein expression levels were determined by western blot. As shown in FIG. 4B, treatment with the DNA damaging agents resulted in increased SIRT4 protein expression in both cell types.

These results indicate that SIRT4 mRNA and protein expression is induced by genotoxic stress.

### Example 4

### SIRT4 Regulates Glutamine Metabolism in Response to Genotoxic Stress

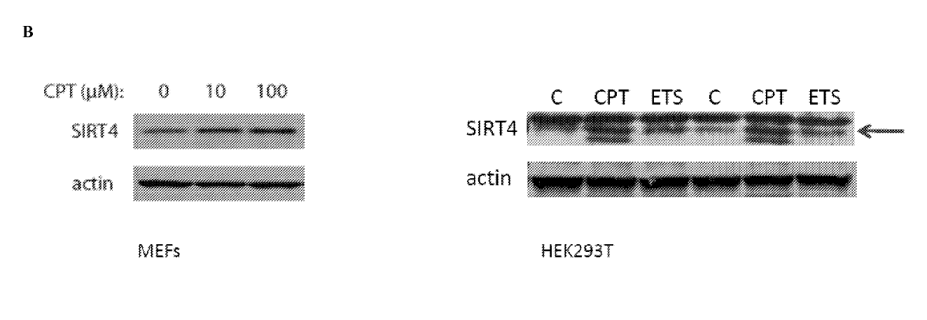

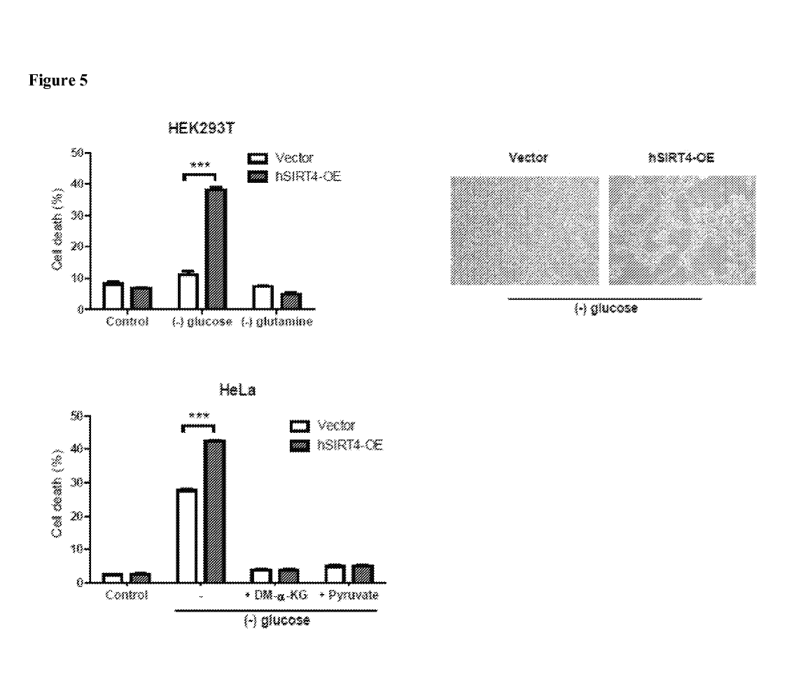

The survival of HEK293T cells or HeLa cells stably over-expressing human SIRT4 (hSIRT4-OE) cultured without glucose or glutamine was examined. Cell death was measured by propidium iodide staining. As shown in FIG. 5, cell death was significantly increased in both cell lines in the absence of glucose. Survival was restored when DM-α-KG or pyruvate was added to the growth medium. These results indicate that SIRT4 regulates glutamine metabolism.

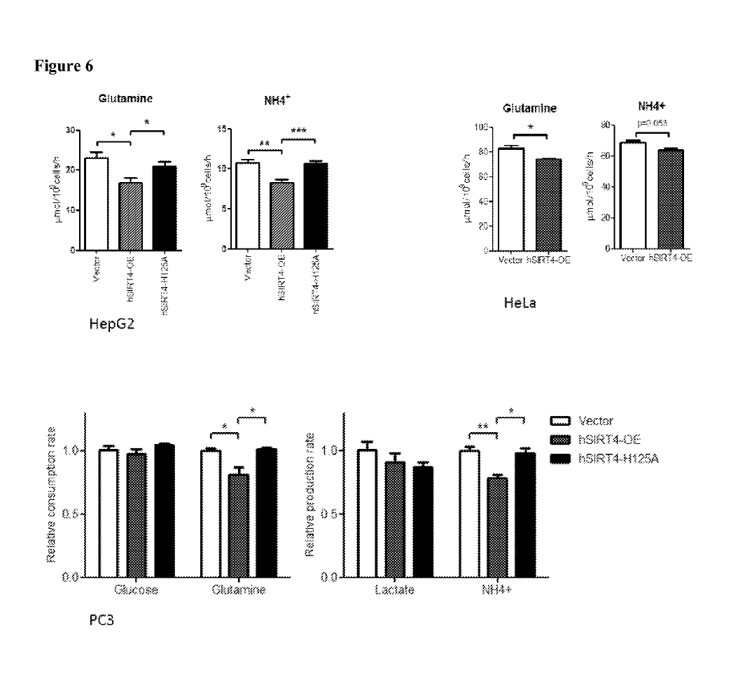

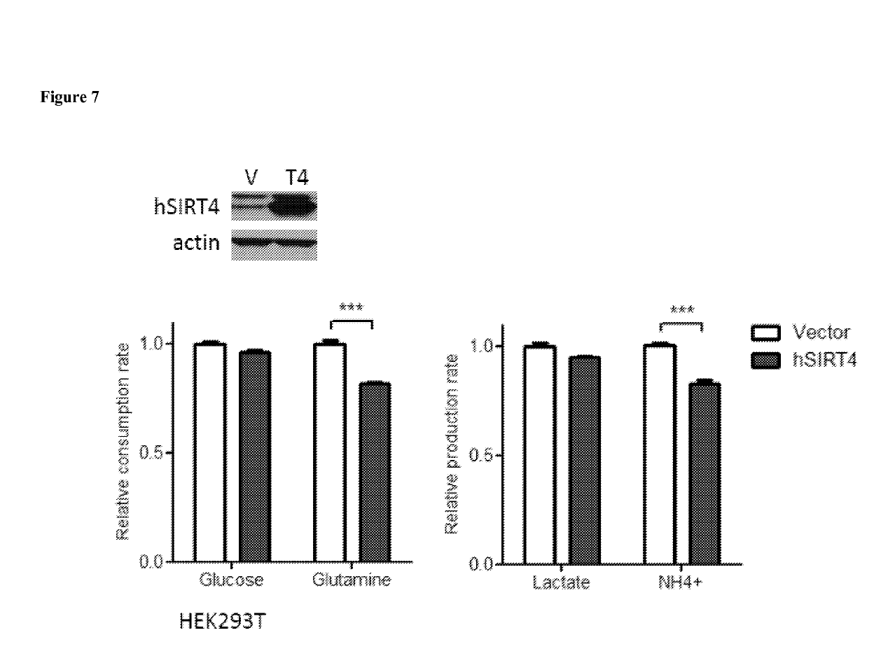

Glutamine metabolite levels were measured in media from HepG2, HeLa and PC3 cells stably expressing human SIRT4 or SIRT4 (H125A) mutant were examined. As shown in FIG. 6, over-expression of human SIRT4 but not SIRT4 (H125A) mutant resulted in decreased levels of glutamine and NH₄⁺ in the growth medium. This further confirms that SIRT4 regulates glutamine metabolism. This result was confirmed with HEK293T cells transiently transfected with human SIRT4 (FIG. 7).

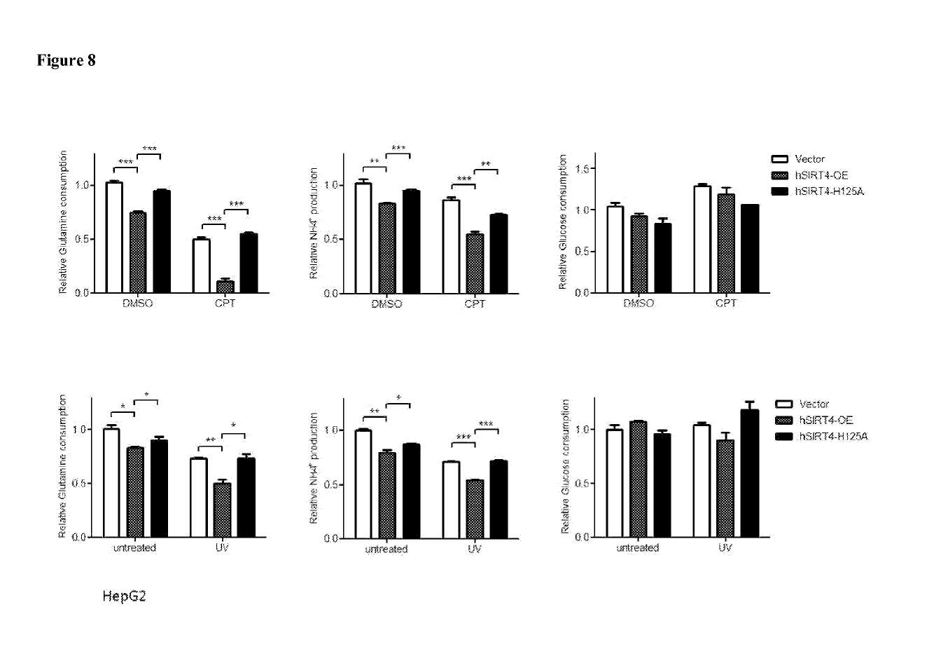

It was next examined whether SIRT4 regulates glutamine metabolism in response to genotoxic stress. HepG2 cells were treated with 14 μM CPT or 50 J/m² UV and metabolite levels were measured as described above. As shown in FIG. 8, over-expression of SIRT4 in DNA-damaging-agent-treated cells resulted significantly reduced levels of glutamine expression and NH₄⁺ production compared to cells that did not over-express SIRT4 or cells that over-express mutant SIRT4. These results indicate that SIRT4 regulates glutamine metabolism in response to genotoxic stress.

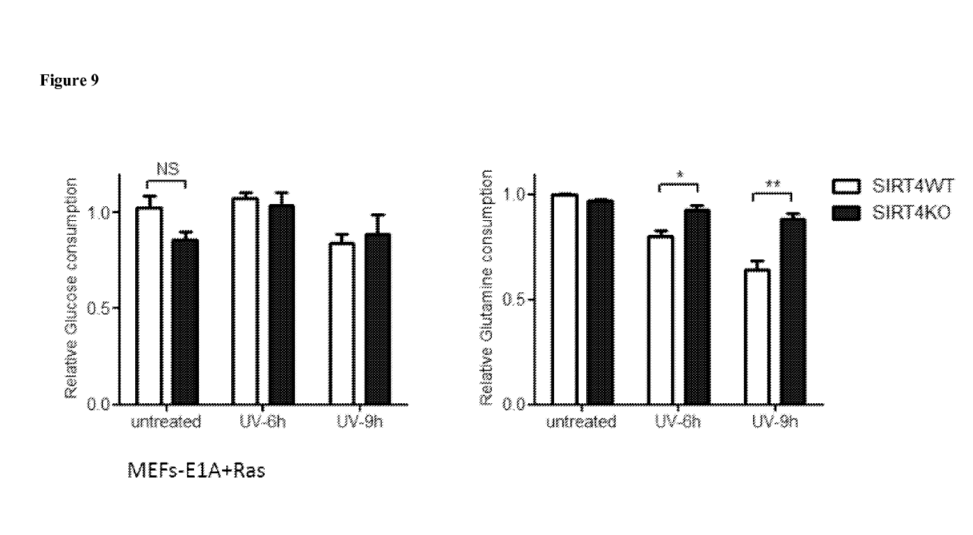

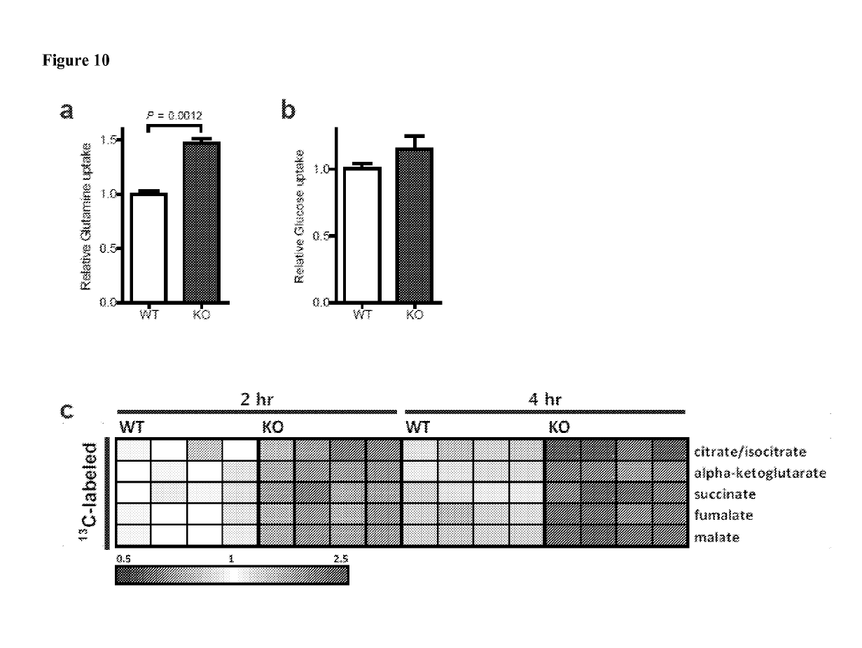

Transformed SIRT4 knockout and wild-type MEF cells were treated with 30 J/m² UV, then metabolite levels were measured using the BioProfile Flex analyzer as described above. As depicted in FIG. 9 and FIG. 10 SIRT4 knock-out (KO) MEFs consumed more glutamine than did wild-type (WT) cells. To test directly whether SIRT4 regulates glutamine-derived anaplerosis, WT and KO MEFs were cultured in medium containing ¹³C-labeled glutamine for 2 or 4 hours and isotopic enrichment of TCA cycle intermediates was measured. Loss of SIRT4 resulted in an increase of ¹³C-labeled metabolites derived from ¹³C-labeled glutamine in all TCA cycle intermediates measured (FIG. 10c). These data suggest that SIRT4 loss drives increased entry of glutamine-derived carbon into the TCA cycle and that SIRT4 is required for repression of glutamine metabolism during the genotoxic stress response.

### Example 5

### SIRT4 Inhibits Mitochondrial Metabolism

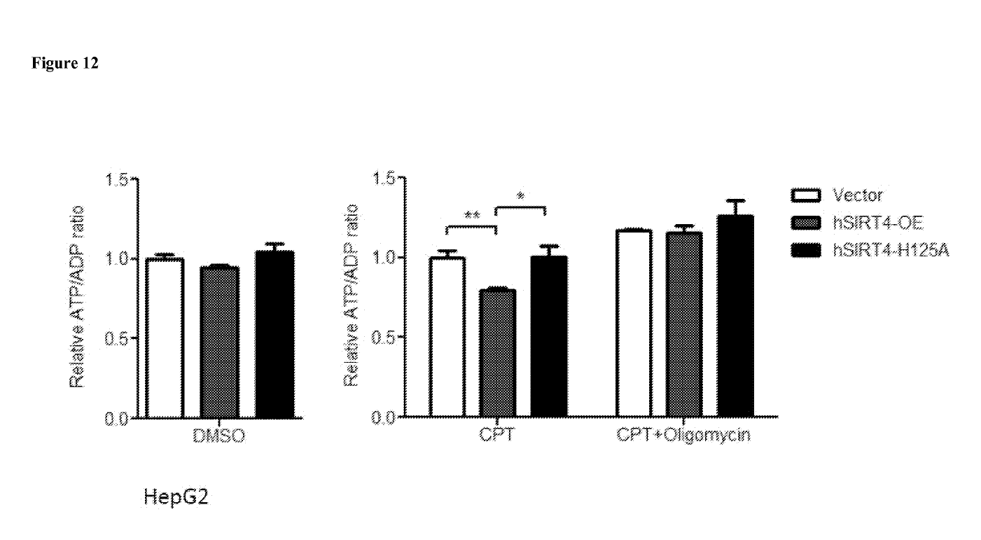

The relationship between SIRT4 and mitochondrial metabolism was next investigated. The relative ATP/ADP ratio of HepG2 cells that stably expressed human SIRT4 or SIRT4 mutant was measured following treatment with DMSO, CPT (14 μM) or CPT+oligomycin (5 g/ml) for 18 hours. HepG2 cells that over-expressed human SIRT4 had a reduced ATP/ADP ration following CPT treatment, but HepG2 cells over-expressing mutant SIRT4 did not (FIG. 12). This difference disappeared when the cells were also treated with oligomycin.

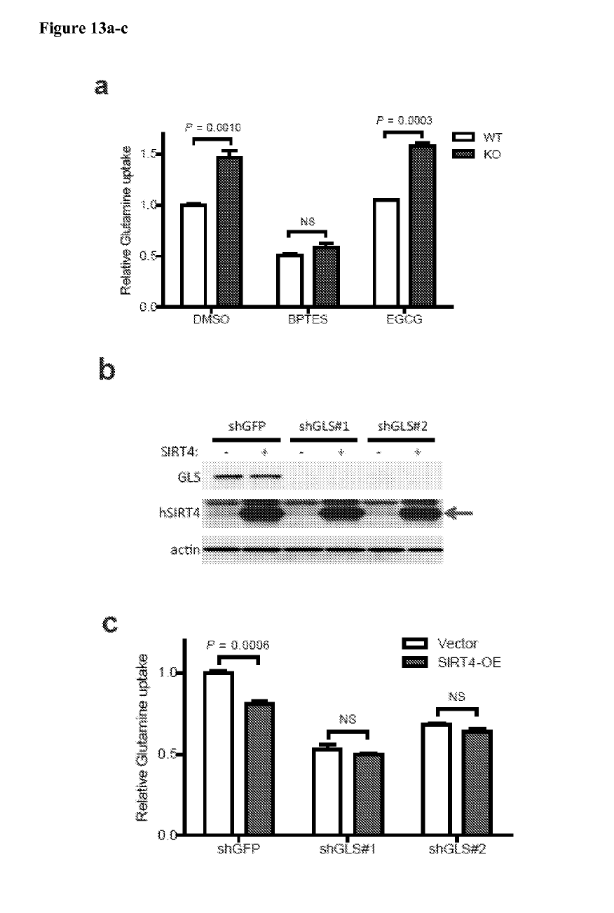

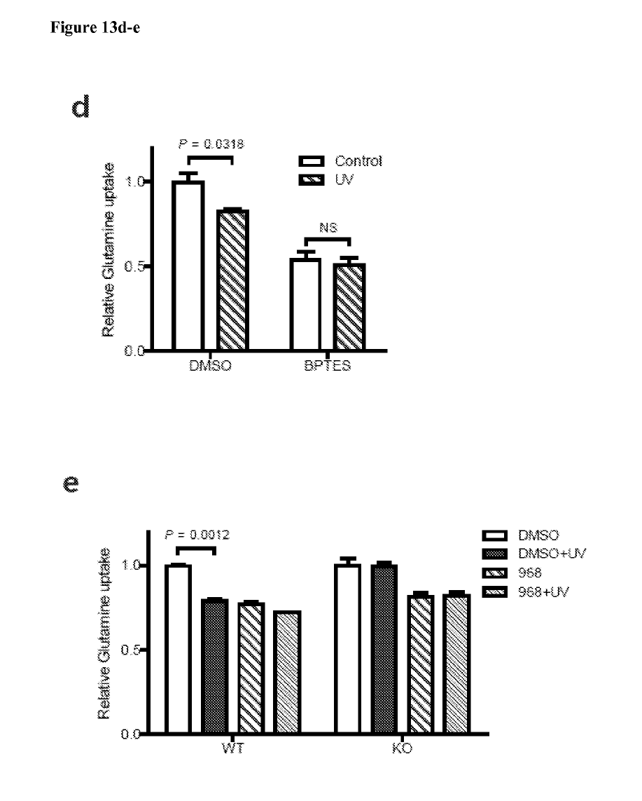

As described above, SIRT4 represses glutamine-derived mitochondrial anaplerosis. This node was investigated further using a pharmacological and genetic approach. GLS is the first required enzyme for mitochondrial glutamine metabolism, and catalyzes the hydrolysis of glutamine to glutamate and ammonia. Using an inhibitor of GLS1, bis-2-(5-phenylacetoamido-1,2,4-thiadiazol-2-yl)ethyl sulfide (BPTES), it was determined that 10 μM BPTES treatment repressed glutamine uptake and rescued the increased glutamine consumption of SIRT4 KO cells (FIG. 13a). Drugs that block glutamate dehydrogenase (GDH) activity were also examined. There were no effects of a GDH inhibitor, 50 μM of Epigallocatechin gallate (EGCG), in glutamine uptake. Moreover, SIRT4 overexpression could no longer inhibit glutamine uptake and ammonia production when GLS1 was targeted using short hairpin RNA (shRNA) (FIG. 13b, c). Finally, cells were treated with chemical inhibitors of mitochondrial glutaminolysis in order to probe the model that suppressing this node could block the repression of glutamine uptake by DNA damage. UV treatment could no longer repress glutamine uptake in the presence of BPTES (FIG. 13d). A similar result was obtained with the compound 968, a small molecule inhibitor of GLS (FIG. 13e). Taken together, these data demonstrate that SIRT4 is a critical regulator of mitochondrial glutamine metabolism during the metabolic response to genotoxic stress.

### Example 6

### SIRT4 Protects Against DNA Damage-Induced Apoptotic Cell Death

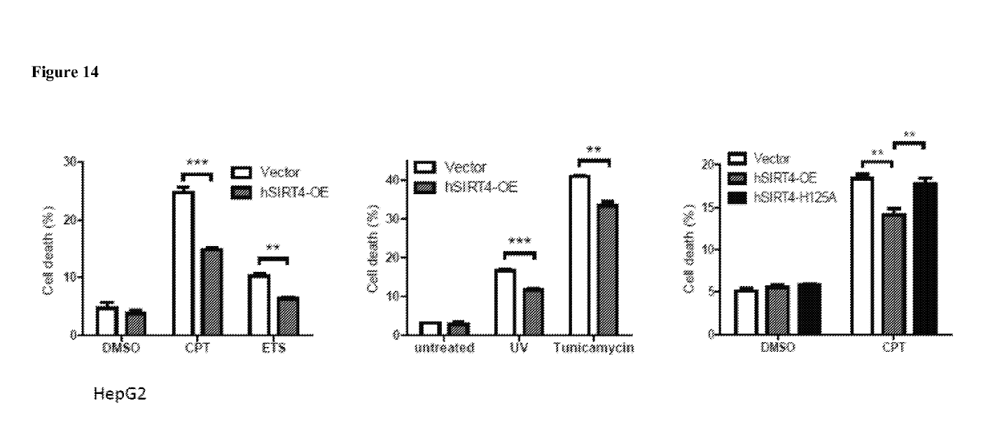

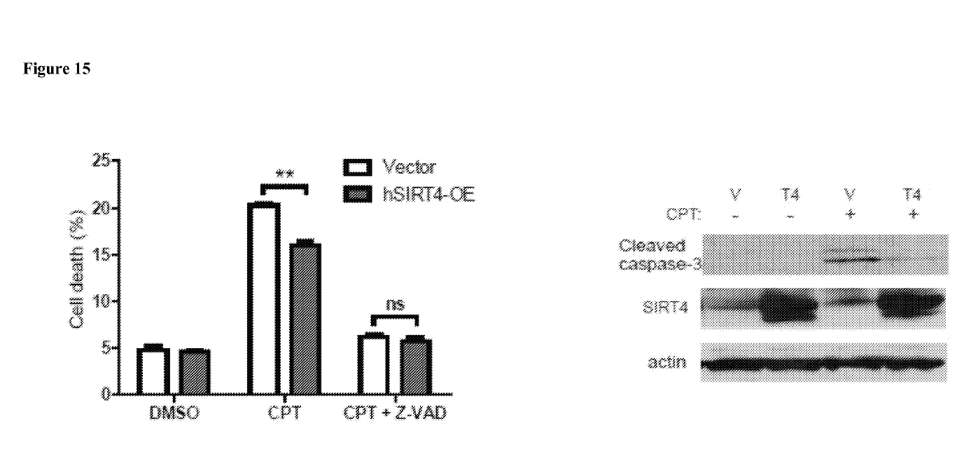

It was next investigated whether SIRT4's inhibition of glutamine metabolism and mitochondrial metabolism would protect cells against apoptotic cell death induced by DNA damage. Survival of HepG2 cells stably expressing human SIRT4 or SIRT4 (H125A) mutant was examined following treatment of the cells with 14 μM CPT, 50 J/m² UV, ETS or 48 μM tunicamycin. Cell death was measured by propidium iodide (PI) staining. As shown in FIG. 14, HepG2 cells that over-expressed SIRT4 had significantly reduced levels of apoptotic cell death upon treatment with the DNA damaging agents. Reduced cell death correlated with reduced levels of cleaved caspase-3 in the SIRT4 over-expressing cells (FIG. 15).

Survival of HepG2 cells stably expressing human SIRT4 was examined following treatment of the cells with CPT or CPT+Z-VAD (100 μM). Cell death was measured by propidium iodide staining. As shown in FIG. 15, HepG2 cells that over-expressed SIRT4 had significantly reduced levels of apoptotic cell death when treated with CPT alone, but not when treated with CPT and Z-VAD.

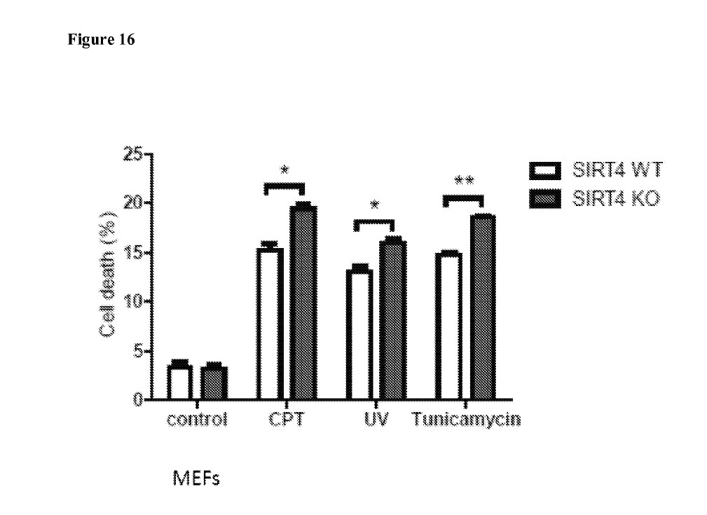

Survival of SIRT4 wild-type and knockout MEF cells was examined following treatment of the cells with CPT, UV or tunicamycin. Cell death was measured by propidium iodide staining. As shown in FIG. 16, MEF cells that lacked SIRT4 had significantly elevated levels of apoptotic cell death when treated with the DNA damaging agents compared to SIRT4 wild-type cells.

Taken together, these results indicate that SIRT4 protects against DNA damage-induced apoptotic cell death.

### Example 7

### SIRT4 Inhibits Cancer Cell Proliferation by Repressing Mitochondrial Glutamine Metabolism

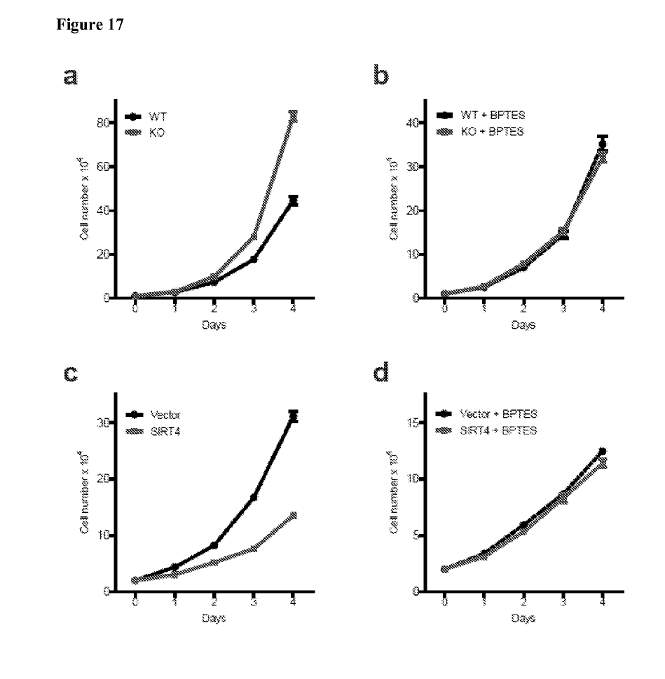

Glutamine is an essential metabolite for proliferating cells, and many cancer cells exhibit a high rate of glutamine consumption. Increased glutamine metabolism suggested that SIRT4 KO MEFs might be using glutamine to support increased proliferation. Indeed, KO cells significantly grew faster than did WT cells (FIG. 17a). To test whether enhanced glutamine metabolism drove the proliferative phenotype of KO cells, cells were cultured with BPTES and proliferation was measured. BPTES completely abrogated the increased proliferation of KO cells (FIG. 17b). In contrast, overexpression of SIRT4 in HeLa cells, which use glutamine as a major energy source, significantly inhibited their growth (FIG. 17c). Control and SIRT4-expressing cells proliferated at similar rates when cultured in media containing BPTES (FIG. 17d). These data suggest that SIRT4 regulates cancer cell growth by regulating the use of glutamine.

### Example 8

### SIRT4 Inhibits Tumorogenesis

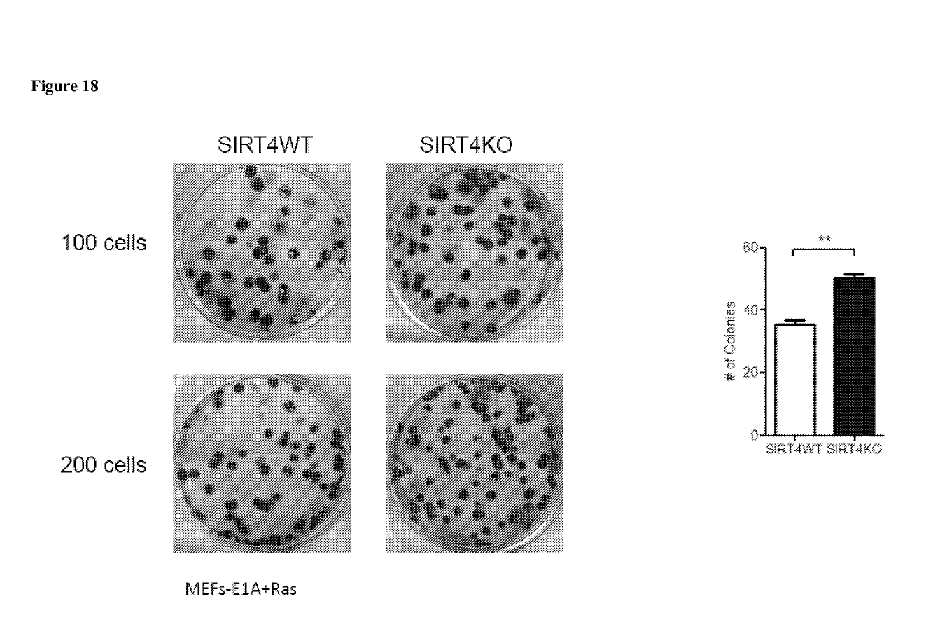

The relationship between SIRT4 and tumorigenesis was next examined. A foci formation assay was performed with transformed SIRT4 wild-type and SIRT4 knockout MEF cells. MEF cells were plated onto a 6 well plate at a concentration of 100 or 200 cells per well, stained with crystal violet at 10 days and counted. As shown in FIG. 18, the SIRT4 knock-out MEF cells formed significantly more colonies than the SIRT4 wild-type MEF cells.

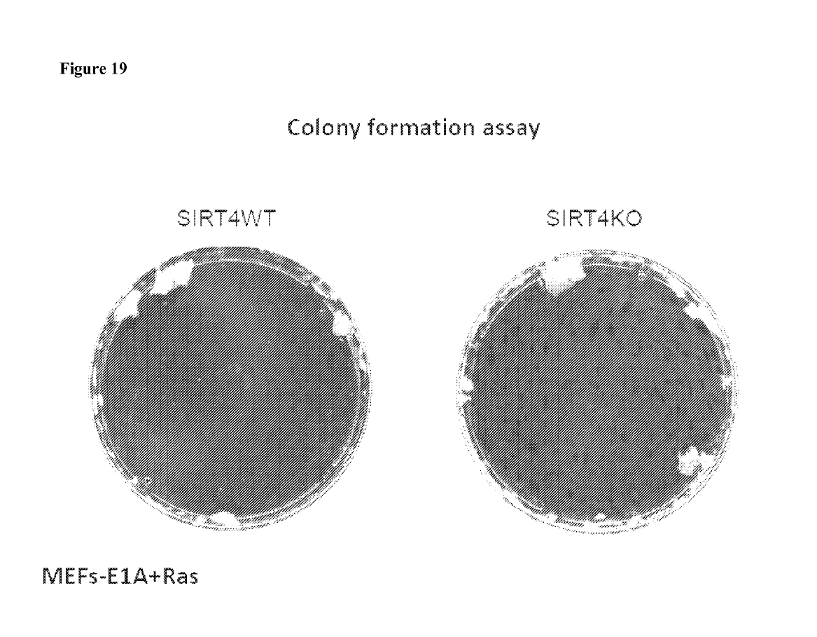

Next, a colony formation assay was performed. SIRT4 wild-type and SIRT4 knockout MEF cells were plated onto a 10 cm plate and medium was replaced every 3-4 days for 28 days and the plates were stained with crystal violet. As shown in FIG. 19, examination of the MEF cells revealed that the SIRT4 knockout cells but not the SIRT4 wild-type cells acquired a tumorigenic phenotype.

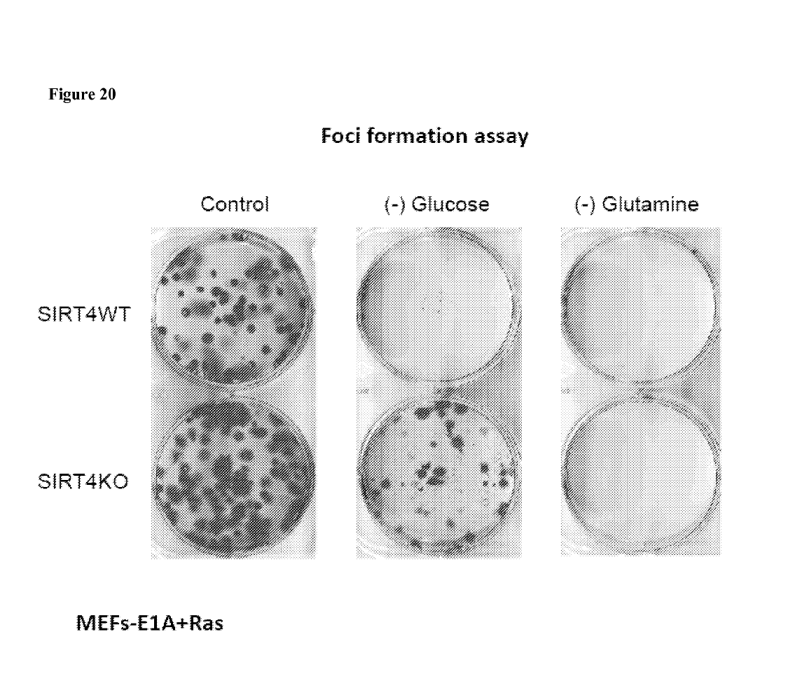

To determine whether glutamine metabolism is essential for the transformative properties of SIRT4 knockout cells, a foci formation assay was performed as described above using 200 cells per well in control growth medium that contained both glucose and glutamine or in growth medium that lacked either glucose or glutamine. As shown in FIG. 20, SIRT4 knock-out MEF cells but not wild-type MEF cells were able to form foci in glucose deficient medium. Neither the SIRT4 knock-out MEF cells nor the wild-type MEF cells were able to form foci on glutamine deficient medium. These results indicate that glutamine metabolism is essential for the transformative properties of SIRT4 knockout cells.

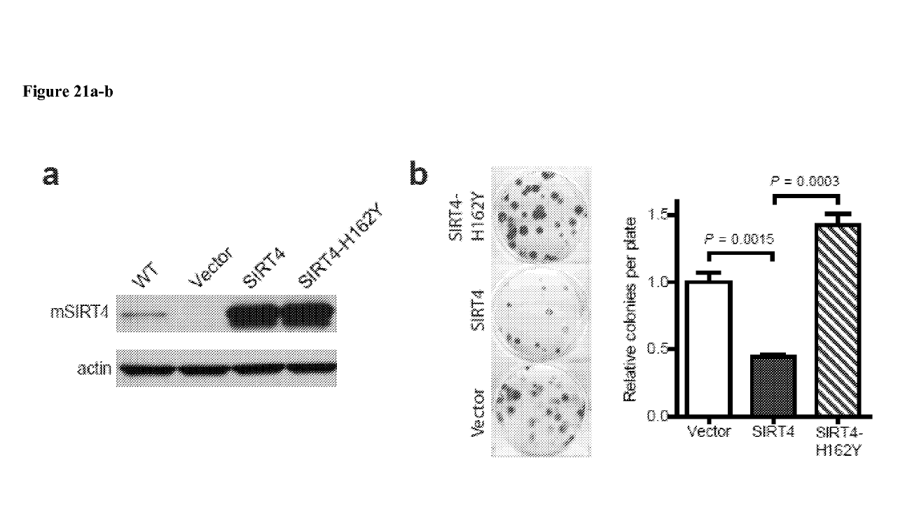

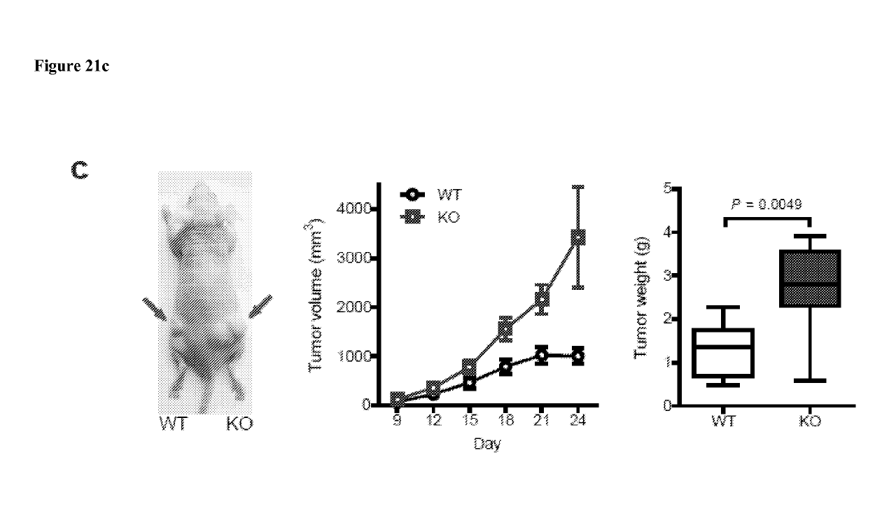

Focus formation assays were performed with transformed KO MEFs reconstituted with SIRT4 or a catalytic mutant of SIRT4. Reconstitution of KO cells with SIRT4 can reverse the phenotype, whereas reconstitution with a catalytic mutant of SIRT4 cannot (FIG. 21a, b).

To extend these findings to in vivo models, xenografts were performed using transformed MEFs. Transformed SIRT4 WT and KO MEFs were injected subcutaneously into nude mice (6- to 8-week old males). Consistent with the in vitro results, it was found that loss of SIRT4 promoted larger tumor volumes and weight compared to WT cells (FIG. 21c).

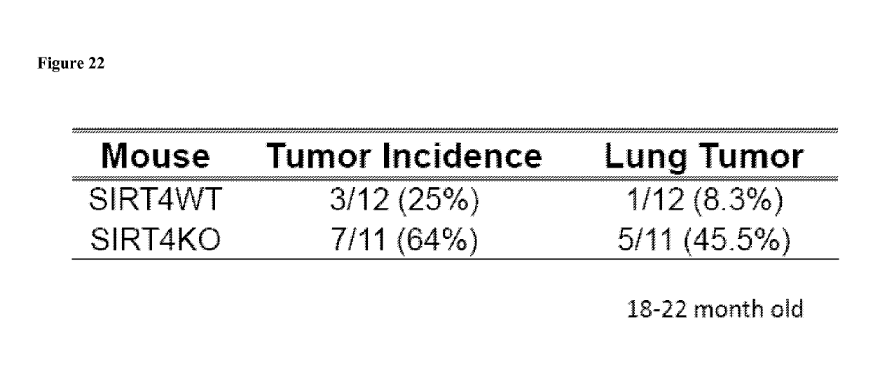

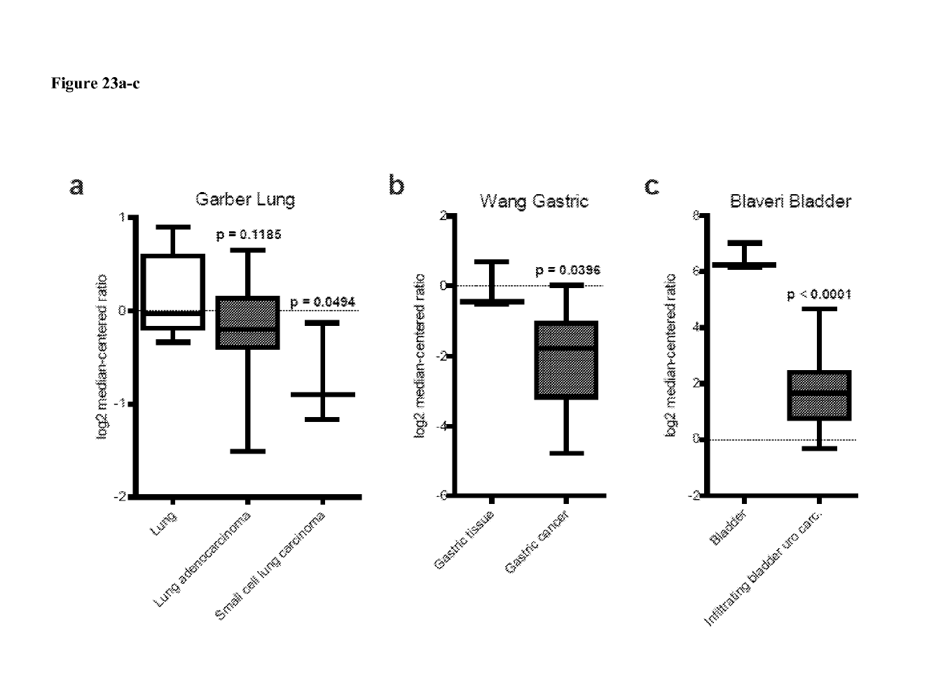

The tumor incidence of SIRT4 knock-out mice was examined. SIRT4 mice had substantially increased tumor incidence in general and lung tumor incidence in particular (FIG. 22). These results indicate that SIRT4 inhibits tumorigenesis.

### Example 9

### SIRT4 Expression is Decreased in Human Cancers

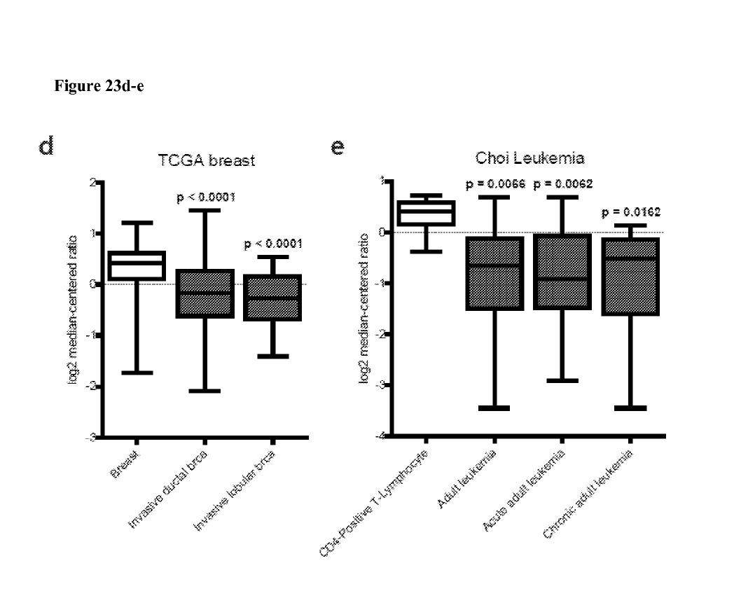

The analysis of Oncomine datasets showed that SIRT4 mRNA level was reduced in several human cancers, such as small cell lung carcinoma, gastric cancer, bladder carcinoma, breast cancer and leukemia (FIG. 23). This suggests that SIRT4 may be a bona fide tumor suppressor.

### EQUIVALENTS

Those skilled in the art will recognize, or be able to ascertain using no more than routine experimentation, many equivalents of the specific embodiments of the invention described herein. Such equivalents are intended to be encompassed by the following claims.

We claim:

1. A method of treating or preventing cancer in a subject comprising administering to the subject an agent that increases the activity or expression of SIRT4.

2. The method of claim 1, wherein the agent is a small molecule, a polypeptide or a nucleic acid.

3. The method of claim 1, wherein the agent is a SIRT4 protein or a fragment thereof.

4. The method of claim 1, wherein the agent is a nucleic acid that encodes a SIRT4 protein or a fragment thereof.

5. The method of claim 1, wherein the agent increases the expression of SIRT4 protein or mRNA.

6. The method of claim 1, wherein in the subject has or is predisposed to lung cancer.

7. The method of claim 1, wherein the agent inhibits glutamine consumption.

8. The method of claim 1, wherein the subject is human.

9. A method of inhibiting the proliferation of a tumor cell comprising contacting the tumor cell with an agent the increases the activity or expression of SIRT4.

10. The method of claim 5, wherein the agent is a small molecule, a polypeptide or a nucleic acid.

11. The method of claim 10, wherein the agent is a SIRT4 protein or a fragment thereof.

12. The method of claim 10, wherein the agent is a nucleic acid that encodes a SIRT4 protein or a fragment thereof.

13. The method of claim 10, wherein the agent increases the expression of SIRT4 protein or SIRT4 mRNA.

14. The method of claim 10, wherein in the tumor cell is a lung tumor cell.

15. The method of claim 10, wherein the tumor is in a subject.

16. The method of claim 10, wherein the subject is human.

17. The method of claim 10, wherein the tumor cell has reduced SIRT4 activity.

18. The method of claim 16, wherein the tumor cell comprises a mutated SIRT4 gene.

19. A method of determining whether a subject is predisposed to cancer comprising the steps of:
a. obtaining a tissue sample from the subject; and
b. determining whether the tissue sample comprises a mutated SIRT4 gene;
wherein the presence of a mutated SIRT4 gene indicates that the subject is predisposed to cancer.

20. The method of claim 19, wherein the tissue sample is selected from the group consisting of a blood sample, a tissue biopsy sample, a cheek swab sample, a hair sample, a saliva sample and a skin sample.

21. The method of claim 19, further comprising determining whether the tissue sample comprises a second mutated SIRT4 gene.

22. The method of claim 19, further comprising determining whether the tissue sample comprises an unmutated SIRT4 gene, wherein the presence of an unmutated SIRT4 gene indicates the subject is not predisposed to cancer.

23. The method of claim 19, wherein the determining step comprises sequencing at least part of the SIRT4 gene.

24. The method of claim 19, wherein the determining step comprises contacting the SIRT4 gene or an amplicon thereof with a nucleic acid probe.
